(19) United States
(12) Patent Application Publication
(10) Pub. No.: US 2023/0049100 A1
(43) Pub. Date: Feb. 16, 2023

(54) METHOD FOR DIAGNOSIS OF CANCER BASED ON QUANTITATIVE BIOMARKERS AND A DATABASE THEREOF

(71) Applicant: QUANTICISION DIAGNOSTICS INC., Chapel Hill, NC (US)

(72) Inventor: Jiandi ZHANG, Chapel Hill, NC (US)

(21) Appl. No.: 17/758,673

(22) PCT Filed: Nov. 2, 2020
(86) PCT No.: PCT/US2020/058582

§ 371 Date: Jul. 12, 2022

Related U.S. Application Data
(60) Provisional application No. 62/929,396, filed on Nov. 1, 2019.
(60) Provisional application No. 62/946,742, filed on Dec. 11, 2019.

(51) Int. Cl. G16H 10/40; G16B 40/00; G16H 50/20; G01N 33/574

(52) U.S. Cl. CPC G16H 10/40; G16B 40/00; G16H 50/20; G01N 33/57415

(57) ABSTRACT

Provided are methods, system and software for diagnosis, prediction and prognosis of a cancer patient based on the quantitative level of a set of biomarkers. Also provided is a database for the purpose of recording the quantitative level of a set of biomarkers.

### CROSS-REFERENCE TO RELATED APPLICATIONS

This application claims the benefit of U.S. provisional application No. 62/929,396, filed on Nov. 1, 2019, which is hereby incorporated in reference by its entirety for all purposes.

### FIELD OF THE INVENTION

The present disclosure relates to methods, system and software for diagnosis, prediction and prognosis of a cancer patient based on the quantitative level of a set of biomarkers. More specifically, this disclosure provides diagnosis, prediction and prognosis of a cancer patient based on the quantitative level of a set of biomarkers when referenced with those same sets in a database.

### BACKGROUND

For the majority of cancer patients, their tumor tissues are surgically removed and archived in Formalin Fixed Paraffin Embedded (FFPE) format at hospitals or other medical institutes. As a result, millions of archived FFPE specimens, accompanied by detailed medical records including treatments administered and ensuing clinical outcomes, have accumulated as an enormous yet underutilized resource. The sheer number of these FFPE specimens allow comprehensive coverage of molecular identities at the individual level. When combined with their known clinical outcomes, these specimens become an unrivaled resource for clinical studies geared towards personalized medicine, where we may identify known cases with similar molecular identities for every single cancer patient in the world.

In clinical practice, immunohistochemistry (IHC) is widely used to assess at protein level of a biomarker for diagnostic or prognostic purpose. A typical IHC report of a biomarker either expresses as “+” or “−”, or it is further categorized as “0, 1+, 2+, 3+”. For example, the expression level of one commonly used biomarker for breast cancer diagnosis, Human epidermal growth factor receptor 2 (Her2), is assessed using IHC to determine if Her2-dependent therapy should be included in the treatment plan. The IHC results are categorized into three groups: 0 and 1+, 2+, and 3+. 0 and 1+ group is considered as negative, 3+ is considered as positive while 2+ is considered as equivocal.

The combination of IHC result is used routinely in clinical practice for diagnosis and prognosis. For example, for breast cancer patients, four biomarkers, including Estrogen receptor (ER), progesterone receptor (PR), Ki67 and Her2 are used to divide patients into four sub-groups of luminal A, luminal B, Her2, triple negative groups. The IHC results from Her2 and ER/PR are used to subgroup patients into Luminal, Her2 and triple negative group, while Ki67 level is used to separate luminal A type from luminal B type.

The categorized results of IHC analysis makes it difficult for clinical practice. For example, while there are significant differences among individual patients with positive results, they are all considered in the same category in clinical practice. Thus, results from IHC analysis are unable to be used for extensive data analysis to provide more accurate, more predictive diagnosis or prognosis.

The IHC method is also severely limited by the inherent subjectivity and inconsistency. The heterogeneity of tumor tissues also complicate the diagnosis process.

There are multiple efforts to measure biomarker at tissue level as absolute and continuous variables. For example, Enzyme linked immunosorbent Assay (ELISA) may be used to measure a biomarker level in fresh and frozen tissues. However, this method is unable to measure biomarker level in FFPE specimens, thus significantly limits its usage in clinical diagnosis and prognosis.

Quantitative Dot Blot (QDB) method is able to measure a biomarker level as absolute and continuous variables in fresh, frozen and FFPE specimens in a high throughput format. Introduction of a protein standard, either in the form of a recombinant protein, or a purified protein, conveniently translates this method into an absolute quantitative assay to measure the absolute content of a specific protein at cellular or tissue level⁶.

Until the development of QDB method, millions of archived FFPE specimens are inaccessible to current available protein techniques due to their inability to differentiate individual FFPE specimen at population level. The current prevailing methods of protein analysis, including immunohistochemistry (IHC), Western blot analysis [4], Reverse phase protein microarrays (RPPA) [5], and mass spectrometry (MS) [6], have all been used to analyze FFPE specimens. Nonetheless, they become inadequate for evaluating the enormous quantity of FFPE specimens.

For both IHC and Western blot analysis, the qualitative nature of their results obscures individual differences at population level.

Other methods may measure protein levels quantitatively to reveal individual differences at the population level, but offer relative results to limit the scale of the study. We may use an example to illustrate this limitation better. The expression level of a protein may be expressed in absolute terms (for example, nmole/g), or in relative terms (% of a reference protein B). While we can compare the protein levels in absolute terms easily across multiple analyses, it is harder to compare the results from analyses with varying levels of reference protein B in each analysis.

This is the case for MS and RPPA, in which results are expressed as values relative to a reference protein, which may vary for each individual study [Boellner, et al, Microarrays, 4(2): 98-114, 2015 ,DeSouza, et al, Clin. Biochem. 46: 421-431, 2013]. Thus, the scale of the studies based on these methods is limited by the number of specimens included in a single study, unable to be expanded upon by incorporating results from other studies. The same issue also holds true to datasets generated from Real-time PCR (RT-PCR) method.

QDB method, on the other hand, may provide a method to accommodate the enormous amount of FFPE specimen datasets of continuous and absolute nature. With respect to continuity, quantitative assessments are needed to distinguish the subtle differences among individual FFPE specimens at population level; with respect to absoluteness, the quantitation of individual proteins should be consistent, regardless of location, timing, etc. to ensure that data can be reliably shared, cross-examined, and combined to offer the much needed growth of the dataset to accommodate the enormous amount of FFPE specimens.

The current invention provides methods, systems and software to aid diagnosis of a patient by relying of enormous amount of archived FFPE specimens worldwide. The adoption of this method in clinical practice may significantly improve the effectiveness of treatment to achieve the goal of personalized medicine.

### SUMMARY OF THE INVENTION

The present invention provides method to provide diagnosis, prediction and prognosis of cancer using three or more biomarkers as continuous variables. The evaluation of biomarkers is quantitatively measured instead of categorized as in current prevailing methods, and expressed in absolute unit to allow easily incorporation into an existing database.

The sample can be a tissue from a subject. In one embodiment of current invention, the tissue refers to a biopsy tissue. In another embodiment of current invention, the tissue refers to a Formalin Fixed Paraffin Embedded specimen (FFPE specimen).

The subject can be a patient. Specifically, the subject can be a cancer patient. In one embodiment of current invention, the subject can be a breast cancer patient.

A retrospective cancer profile (RC) database, or more accurately, databases of different cancer types (breast, colorectal, or prostate cancer) are developed based on absolutely quantified protein biomarkers to take full advantage of the vast amount of archived FFPE specimens.

When measured quantitatively and absolutely, combinations of a plurality of protein biomarkers are sufficient to differentiate individual FFPE specimen from millions of archived FFPE specimens. In certain sense, the combination of these protein biomarkers becomes a unique “fingerprint” for each FFPE specimen in the database.

This unique “fingerprint” is used as a nucleus to anchor the matching clinical records, including both the traditional clinicopathological factors, the treatments administered, and the ensuing clinical outcomes, for a holistic picture of each FFPE specimen.

All the information from these various aspects constitutes an individual cancer profile (ICP) for every FFPE specimen in the database. Any other clinical-related trait may also be included in these cancer profiles. For example, genetic information including small nucleotide variations (SNV), chromosomal alterations, and scores of various genetic predictor assays, can all be included in the cancer profiles.

The absolute nature of the database ensures the continuing growth of the database. ICPs, although from different sources, may be combined efficiently due to the absoluteness of the data. New cancer profiles will also be added and supplemented over the time. Over the years, this database is expected to accommodate a fair share of these archived FFPE specimens to support the “big data” supported clinical diagnosis.

Additionally, the above-described method further includes a method of generating a RC database to provide diagnosis, prediction, or prognosis of cancer, comprising: providing a plurality of subjects each having a known clinical outcome of a cancer; generating an ICP from each of the plurality of subjects, the ICP comprising i) a plurality of protein biomarkers measured absolutely and quantitatively, and ii) a known clinical outcome of a cancer; and storing the generated ICPs of the plurality of subjects in the database.

In one embodiment of current invention, the expression level of a biomarker can be measured as absolute and continuous variables.

In one embodiment of current invention, the expression level of a biomarker can be measured using Mass spectrometry.

In one embodiment of current invention, the expression level of a biomarker can be measured using Enzyme linked Immunosorbent Assay (ELISA).

In one embodiment of current invention, the expression level of a biomarker can be measured using QDB method,

In one embodiment of current invention, the protein levels of three or more biomarkers can be measured by any combination of ELISA, QDB and Mass Spectrometry.

The quantitated level of a biomarker from ICP of the database can be combined with its relevant clinical information for mathematical analysis for medical use. For example, the putative association between absolute level of a biomarker and the disease free survival “DFS” can be explored to provide predictive clinical prognosis for a patient.

In one embodiment of current invention, the levels of a plurality of biomarkers as continuous variables from an ICP can be combined with relevant clinical information, including, but not limited to, the disease free survival, the overall survival, the side response, the age, the progression stage of the disease, to find a causal relationship, and this association can be used for diagnosis and prognosis purpose.

In one embodiment of current invention, the absolute levels of three or more biomarkers from an ICP can be used as coordinates of (x, y, z) to locate a subject in a space determined by X, Y and Z axes (the spot). The spot of the sample is combined with relevant clinical information, including, but not limited to, the disease free survival, the overall survival, the side response, the age, the progression stage of the disease, to find a spatial association, and this association can be used for diagnosis and prognosis purpose.

In one embodiment of current invention, the spots of more than one ICP in the space created by X, Y and Z axes can be grouped as a relevant clinical sub-group to be associated with clinical diagnosis, prediction and prognosis.

Another aspect of the present invention relates to a reference database for diagnosing cancer in a patient based on quantitative analysis of more than one marker in a biopsy sample from the patient. The reference database includes a plurality of ICPs. each of the plurality of ICP is prepared by steps of: (a) providing a biopsy sample from a cancer patient with a known diagnosis; (b) measuring three or more said markers as absolute and continuous variables in the biopsy sample; (c) locating each ICP in a space using values of three biomarkers as x, y, z in a space (the spot); (d) associate each ICP by the spot with the known diagnosis, prediction and prognosis of the cancer patient thereby obtaining a reference profile by spatial localization.

Yet another aspect of the present invention is directed at a method for diagnosing cancer in a patient. The method includes steps of (i) providing a reference spatial database described above, (ii) obtaining a biopsy sample from the patient; (iii) measuring three biomarkers as absolute and continuous variables, the measured result being a continuous variable of three markers in the biopsy sample; (iv) locating the sample in the reference spatial database using values of three marker as (x, y, z); and (v) identifying a reference spatial profile in the reference spatial database that has the best match and outputting the known diagnosis associated with the identified reference profile.

Yet another aspect of the present invention relates to a database for providing diagnosis, prediction, or prognosis of cancer comprising a plurality of ICPs, each generated from a subject having a known clinical outcome of a cancer. Further, an ICP includes i) a plurality of clinical parameters quantitatively measured from an archived FFPE specimen of the subject and ii) a known clinical outcome of a cancer. Furthermore, each of the plurality of clinical parameters represents a quantitative measurement of a biomarker and additionally, the quantitative measurement is continuous and is an absolute amount of the biomarker in the specimen.

Yet another aspect of the present invention relates to a method of providing diagnosis, prediction, or prognosis of cancer in a patient. The method includes: 1) collecting a FFPE specimen of the patient; 2) obtaining from just-described above database: i) the stored ICPs and ii) the set of clinical parameters used in the database; 3) comparing the quantitative level of the set of clinical parameters in the database with those measured from the FFPE specimen of the patient; 4) identifying an ICP from the database that best matches the patient based on the comparison; 5) outputting a clinical outcome of the identified ICP from the database.

In the just-described above method, the comparison is to determine maximum similarity between the set of clinical parameters of an ICP and those of the same set measured from the FFPE specimen of the patient.

In one embodiment of current invention, the similarity can be achieved by comparing a set of protein biomarkers similarity based on their absolute levels. The quantitative level of a biomarker from the patient is used to identify ICPs within a pre-set range of the same biomarker. ICPs with every biomarker of a set biomarkers within the respective preset range of the corresponding biomarker of the same set of biomarkers of the patient are considered similar to the patient.

In one embodiment of the current invention, the preset range of individual biomarker of the set of biomarkers may be the same.

In another embodiment of the current invention, the preset range of a biomarker maybe different from that of another biomarker within the set of protein biomarkers used for assessing similarity between an ICP and the patient.

In one embodiment of current invention, the similarity can be calculated based on the Euclidean distance between the two sets of quantitative clinical parameters.

A plurality of ICPs may be identified from the database based on their similarity, and their clinical outcomes are analyzed mathematically to provide the personalized prognosis for the patient.

A plurality of ICPs may be identified from the database based on their similarity, and the treatment received and the corresponding outcomes maybe analyzed mathematically to identify the treatment plan with the best prognosis for the patient.

In yet another embodiment of current invention, other clinical traits including traditional clinical factors like age, tumor size, tumor grade and node statuses, maybe used to further improve the similarity of an ICP with the patient.

The details of the invention are set forth in the drawing and the description below. Other features, objects, and advantages of the invention will be apparent to those persons skilled in the art upon reading the drawing and the description, as well as from the appended claims.

### BRIEF DESCRIPTION OF THE DRAWINGS

FIG. 1: Using expression levels of PR, ER and Her2 from 1049 breast cancer FFPE specimens as coordinates to create a 3D scatterplot. The expression levels of PR, ER, and Her2 were measured using QDB method, and the values were used to create a 3D scatterplot using Origin software using X axis to present PR, Y axis to present ER and Z axis to present Her2. The distributions of individual spot from each patient divide the space into separate regions, including Hormone group, where samples spread exclusively at the floor by X and Y axes, the Her2 group which wrapped around the Z axis, and the corner group, where samples accumulated at the intersections of X, Y and Z axes. The corner group include both the triple negative group and normal like group group.

FIG. 2: charts showing comparison of the OS of the similarity groups of five hypothetical patients identified based on the absolute levels of ER, PR, Her2 and Ki67, with that of corresponding clinical subtype using Kaplan-Meier survival analysis. The profiles in the database were subtyped into Luminal A-like, Lumina B-like, Her2 positive and Triple negative (TNBC) based on IHC-based surrogate assay , and their OS were used as references to those of the similarity groups to five hypothetical patients using Log Rank test, with p<0.05 as statistical significant. (a), Comparisons of the OS of similarity group to both #1388 & #1843 with that of TNBC subtype; (b), Comparison of the similarity group to #1445 with that of Her2 positive subtype; (c), Comparison of the similarity group to ##1807 with that of Luminal A-like subtype; and (d), Comparison of the similarity group to #1519* with that of Luminal B-like subtype. Biomarkers levels lower than 2XLimit of Quantitation (LOQ) were considered the same to increase number of profiles for analysis.

FIG. 3: charts showing comparison of the OS of the similarity groups of five hypothetical patients identified based on the absolute levels of ER, PR, Her2, Ki67 and cyclinD1, with that of the corresponding clinical subtypes using Kaplan-Meier survival analysis. The profiles in the database were subtyped into Luminal A-like, Lumina B-like, Her2 positive and Triple negative (TNBC) based on IHC-based surrogate assay, and their OS were used as references to those of the similarity groups to five hypothetical patients using Log Rank test, with p<0.05 as statistical significant. (a), Comparisons of the OS of similarity group to both #1388 & #1843 with that of TNBC group; (b), Comparison of the similarity group to #1445 with that of Her2 positive subtype; (c), Comparison of the similarity group to #1807 with that of Luminal A-like subtype; and (d), Comparison of the similarity group to #1519* with that of Luminal B-like subtype. Biomarkers levels within 2XLimit of Quantitation (LOQ) were considered the same to increase number of profiles for analysis.

FIG. 4: figures illustrating evaluation of the OS of profiles receiving different treatments within similarity groups to the five hypothetical patients based on absolute levels of ER, PR, Her2 and Ki67 using Kaplan Meier survival analysis. Profiles with unknown treatment were not included in the analysis. (a), OS analysis of profiles receiving Chemotherapy (Chemo), Endocrine therapy (ET) and both (CET) within the similarity group to #1388; (b), OS analysis of profiles receiving Chemo, ET and CET within the similarity group to #1843; (c), OS analysis of profiles receiving Chemo within the similarity group to #1807; (d), OS analysis of profiles receiving Chemo and CET within the similarity group* to #1519. Biomarkers levels within 2XLimit of Quantitation (LOQ) were considered the same to increase number of profiles for analysis.

### DETAILED DESCRIPTION

Before the present methods are described, it is to be understood that this invention is not limited to particular method and apparatus described, as such may, of course, vary. It is also to be understood that the terminology used herein is for the purpose of describing particular embodiments only, and is not intended to be limiting, since the scope of the present invention will be limited only by the appended claims.

Unless otherwise defined in this disclosure, all technical and scientific terms used herein have the same meaning as is commonly understood by one of skills in the art to which this disclosure belongs.

The subject methods are useful primarily for diagnostic purposes. Thus, as used herein, the terms “determining,” “measuring,” and “assessing,” and “assaying” are used interchangeably and include both quantitative and qualitative determinations. These terms can also refer to both quantitative and semi-quantitative determinations and as such, the term “determining” is used interchangeably herein with “assaying,” “measuring,” and the like. Where a quantitative determination is intended, the phrase “determining an amount” of an analyte and the like is used. Where either a quantitative and semi-quantitative determination is intended, the phrase “determining a level” of an analyte or “detecting” an analyte is used.

“Quantitative” assays in general provide information on the amount of an analyte in a sample relative to a reference (control), and are usually reported numerically, where a “zero” value can be assigned where the analyte is below the limit of detection (LOD).

The terms “subject,” “host,” “patient,” and “individual” are used interchangeably herein to refer to any mammalian subject for whom diagnosis or therapy is desired, particularly humans.

The “spatial” and 3D are interchangeable to describe assigning a spot representing a sample in a 3 Dimensional space, with the intensity of the spot representing the level of a fourth biomarker as continuous variables.

The quantitatively measurement of the protein expression level of a biomarker can be achieved at tissue level using any method. The method is to be considered to its broadest context as long as the expression level of a biomarker is quantified as continuous variables. The method may include, but not limited to, a Mass spectrometry method or an immunoassay method, or in combination of both

The result from current invention can be relative, or, in combination with a protein standard, to be absolute. The terms “relative” and “absolute,” referring to two ways to take a measurement, should be taken into their broadest context. While relative measurement is measuring one thing compared to another thing, absolute measurement is measuring things in known amounts with standard units. Perhaps, the most significant difference between these two measurements lies in each's applicable scope. A relative result is only meaningful under the same experimental setting, while an absolute result should be comparable across a number of different analyses, even analyses taken at vastly separate places or times.

A “sample” or “patient sample” or “specimen” or “biological sample,” which is used interchangeably herein, generally refers to a sample which may be tested for a particular molecule, preferably a specific marker molecule associated with a biological signature, such as a biomarker shown in the paragraph below. Samples may include, but are not limited to, peripheral blood cells, CNS fluids, serum, plasma, buccal swabs, urine, saliva, tears, pleural fluid and the like. A sample used in the present invention generally refers to a tissue.

The term “marker” or “biomarker” here is to be defined at its broadest context. A “marker” or “biomarker,” which is used interchangeably herein, generally refers to a molecule (e.g., a polypeptide) which is differentially present in a sample taken from a subject of one phenotypic status (e.g., having a disease) as compared with that from another phenotypic status (e.g., not having the disease or having a different disease). A biomarker is thus often established if differentially present between two different phenotypic statuses, when the mean or median level of the biomarker in a first phenotypic status relative to a second phenotypic status is calculated to represent statistically significant differences.

In the present invention, the biomarker is a molecule measurable related with a biological or a disease state. It can be well established diagnostic biomarkers (for example, a diagnostic biomarker for IHC), or it can be biomarkers newly identified for in vitro diagnostics.

The terms “reference” and “control” are used interchangeably to refer to a known value or set of known values against which an observed value may be compared. The known value represents an understood correlation between two parameters, e.g., a level of expression of a marker and its associated phenotype. As used herein, the known value constitute a reference profile in a reference database.

Accordingly, a reference database can be prepared storing a number of reference profiles for diagnostic purpose, each recording a marker level of a sample obtained from a subject with either a known diagnosis or known clinical outcome after therapy.

In one embodiment, the present invention related to the generation of a RC database to provide diagnosis, prediction, or prognosis of cancer, comprising: providing a plurality of subjects each having a known clinical outcome of a cancer; generating an ICP from each of the plurality of subjects, the ICP comprising i) a plurality of protein biomarkers measured absolutely and quantitatively, and ii) a known clinical outcome of a cancer; and storing the generated ICPs of the plurality of subjects in the database.

In addition to the multiple protein biomarkers included in individual ICP, other clinical traits, including age, tumor size, tumor grade and node statuses may also be included. Results from clinical assays, including levels of blood biomarkers, and various enzymatic levels may also be included in the ICP.

The current invention is also related to a method to determine a patient profile that best matches one or more reference ICPs in the RC database. The method includes steps of (a) comparing, on a suitably programmed computer, the expression levels of a set of protein biomarkers of the patient with those of individual ICPs in the database; (b) identifying, on a suitably programmed computer, an ICP shares high similarity to the patient ; and (c) outputting to a user interface device, a computer readable storage medium, or a local or remote computer system; or displaying, the maximum similarity or the associated phenotype of the ICPs in the database that best matches the patient profile.

There are multiple methods to assess the similarity of the patient to an ICP in the database, including assessment based on mathematical analysis of a preset of protein biomarkers, or stepwise selection of ICPs by the expression levels of a set of biomarkers.

The expression levels of a biomarker maybe normalized in the mathematical analysis of the similarity of an ICP to the patient, or it may be used without normalization in the analytical process.

The expression level of a biomarker maybe weighted in the analytical process of the similarity of an ICP to the patient, or it may be not weighted in the analytical process of the similarity of an ICP to the patient.

In one embodiment, the similarity is achieved through calculating the Euclidean distance of an ICP to the patient based on a set of protein biomarkers.

The ICPs of similarity to the patient may be identified through stepwise selection based on the expression levels of a set of biomarkers. The method may include a), identify all ICPs of biomarker a within a preset range of that of the patient; b); among selected ICPs, further identifying ICPs with biomarker b within a preset range of that of the patient; c) among further selected ICPs, further identifying ICPs with biomarker c within a preset range of that of the patient; n), among further selected ICPs, further identifying ICPs with biomarker n within a preset range of that of the patient.

The preset range for each biomarker maybe identical or it may be different by each biomarker within the pre-set biomarkers.

In one embodiment of the current invention, the selected ICPs through the above-mentioned process maybe further selected by other clinical traits, including age, sex, tumor size, tumor grade etc. For example, for a male lung cancer patient of age 59, with tumor size 2, tumor grade III, and N2 patients, the ICPs of similarity based on protein biomarkers maybe further narrowed to limit those of similar age (55˜60), male, tumor size 2, tumor grade III and N2 to achieve better diagnosis for the patient.

One method based on spatial relationship as a reference point could include step (a) measuring a sample of three or more biomarkers as continuous variables; (b) using values from three biomarkers (A, B, C) as coordinates (x, y, z) to assign a spot (the spot) representing the sample in the space created by X, Y and Z axes . (c), evaluating the patient based on the spot within the space, particularly, step (c) includes diagnosis and prognosis of a cancer for the patient. Examples of diagnosis and prognosis of a cancer for the patient include disease-free survival, overall survival, or treatment prediction for the cancer.

In the above method, (d) a fourth biomarker (D) may be used to replace one of the above three biomarker, for example, (A, B, D) to assign a spot representing the sample in a new space, and (e) further evaluating the patient based on the spot within the new space, particularly, step (e) includes diagnosis and prognosis of a cancer for the patient. Examples of diagnosis and prognosis of a cancer for the patient include disease-free survival, overall survival, or treatment prediction for the cancer.

Further in the above method, (d) fourth and fifth biomarkers (D & E)) may be used to replace one of the above three biomarker, for example, (A, D, E) to assign a spot representing the sample in a new space, and (e) further evaluating the patient based on the spot within the new space, particularly, step (e) includes diagnosis and prognosis of a cancer for the patient. Examples of diagnosis and prognosis of a cancer for the patient include disease-free survival, overall survival, or treatment prediction for the cancer.

Furthermore in the above method, (d) fourth, fifth and sixth biomarkers (D, E & F)) may be used to assign a spot representing the sample in a new space, and (e) further evaluating the patient based on the spot within the new space, particularly, step (e) includes diagnosis and prognosis of a cancer for the patient. Examples of diagnosis and prognosis of a cancer for the patient include disease-free survival, overall survival, or treatment prediction for the cancer.

The spot in space determined by A, B, C and the new spot determined by A, B, D can be used sequentially to further evaluating the patient, includes diagnosis and prognosis of a cancer for the patient. Examples of diagnosis and prognosis of a cancer for the patient include disease-free survival, overall survival, or treatment prediction for the cancer.

The spot in space determined by A, B, C and the new spot determined by A, B, D can be used in parallel to further evaluating the patient, includes diagnosis and prognosis of a cancer for the patient. Examples of diagnosis and prognosis of a cancer for the patient include disease-free survival, overall survival, or treatment prediction for the cancer.

The spot in space determined by A, B, C and the new spot determined by A, D, E can be used sequentially to further evaluating the patient, includes diagnosis and prognosis of a cancer for the patient. Examples of diagnosis and prognosis of a cancer for the patient include disease-free survival, overall survival, or treatment prediction for the cancer.

The spot in space determined by A, B, C and the new spot determined by A, D, E can be used in parallel to further evaluating the patient, includes diagnosis and prognosis of a cancer for the patient. Examples of diagnosis and prognosis of a cancer for the patient include disease-free survival, overall survival, or treatment prediction for the cancer.

The spot in space determined by A, B, C and the new spot determined by D, E, F can be used sequentially to further evaluating the patient, includes diagnosis and prognosis of a cancer for the patient. Examples of diagnosis and prognosis of a cancer for the patient include disease-free survival, overall survival, or treatment prediction for the cancer.

The spot in space determined by A, B, C and the new spot determined by D, E, F can be used in parallel to further evaluating the patient, includes diagnosis and prognosis of a cancer for the patient. Examples of diagnosis and prognosis of a cancer for the patient include disease-free survival, overall survival, or treatment prediction for the cancer.

In one embodiment, the present invention also includes a method of determining a patient profile that best matches one of a subgroup of reference spatial profile in a reference spatial database. The method includes steps of (a) comparing, on a suitably programmed computer, the localization (the spot) of a sample from a patient in the space using levels of three biomarkers as coordinates, and (b) compare with sub-groups of reference spatial profiles in a reference spatial database to determine the closeness to a sub-group of reference spatial profiles; (b) identifying, on a suitably programmed computer, a sub-group reference spatial profile in a reference database that closest to the spot; and (c) outputting to a user interface device, a computer readable storage medium, or a local or remote computer system; or displaying, the maximum similarity or the associated phenotype of the sub-group of reference spatial profiles in the reference database that best matches the patient spatial profile.

The putative association between the spot of the patient and a clinical trait is explore using a mathematical method. The examples include, but not limited to, the spot in a 3D scatterplot by expression levels of ER, PR and Her2, with the disease free survival of the patient.

This information may provide prognosis for other patient in the same analysis.

The RC database mentioned above may be used to explore the relationship between an ICP and a clinical outcome. A causal relationship maybe explored using mathematical analysis of the expression level of a biomarker with a known clinical outcome associated with each ICP.

The clinical “trait” and “information” are interchangeable, are to be considered in its broadest context. The trait may include, but not limited to, age, sex, blood pressure, glucose level, cancer stage, disease free survival, or any information relevant to the diagnosis, prevention, treatment of the patient.

The clinical outcomes of the ICPs identified from the database may be pooled together for statistical analysis to provide personalized diagnosis, prediction and prognosis for the patient. The methods include, but not limited to, the univariate survival analysis, the multivariate survival analysis, the C-index analysis, the Kaplan-Meier survival analysis, and the Log rank survival analysis.

Distant from the current prevailing subtyping method utilized for breast cancer and prostate cancer diagnosis, the current invention centers on individual patient to identify a group of ICPs highly similarity to the very cancer patient, and analyze their clinical outcomes for the personalized diagnosis, prediction and prognosis to every cancer patient taking advantage of the vast amount of archived FFPE specimens available worldwide.

Clearly, the most archived FFPE specimens available, the more accurate diagnosis, prognosis and prediction would be to the patient.

To put it simply, the current diagnostic method is to draw a few circles, and fit every cancer patient into these few circles to provide precision treatment to the cancer patients. The current invention, on the other hand, use the patient as center of the circle to include ICPs of similarity in a personalized circle to the patient. Consequently, there are as many circles as many cancer patients to allow personalized diagnosis, prediction and prognosis to every cancer patient using the RC database.

It is to be understood that the exemplary embodiments described herein are that for presently preferred embodiments and thus should be considered in a descriptive sense only and not for purposes of limitation. Descriptions of features or aspects within each embodiment should typically be considered as available for other similar features or aspects in other embodiments.

### EXAMPLE 1

### Materials and Methods

Human subjects and human cell lines Formalin fixed paraffin embedded (FFPE) slices were obtained from local hospitals (Yuhuangding Hospital and Binzhou Medical University at Yantai, Shandong,China) together with their clinical information.

General reagents. All general reagents used for cell culture were purchased from Thermo Fisher Scientifics (Waltham, Mass., USA) including cell culture medium and culture dishes. The protease inhibitors were purchased from Sigma Aldrich (St. Louis, Mo., USA). All other chemicals were purchased from Sinopharm Chemicals (Beijing, P. R. China).

Preparation of Lysates. For Formalin fixed paraffin embedded (FFPE) blocks, two 2×15 □m slices were collected into an Eppendorf tube. The slices were de-paraffined and processed in 300□1 lysis buffer (50 mM HEPES, 137 mM NaCl, 5 mM EDTA, 1mM MgCl, 10 mM Na₂P₂O₇, 1%TritonX-100, 10% glycerol) with protease inhibitors (2□g/ml Leupeptin, 2□g/ml Aprotinin, 1□g/ml pepstatin, 2mM PMSF, 2 mM NaF) with sonication for 2 mins before they were centrifuged at 12000×g for 5 mins. The supernatants were collected for immunoblot analysis. The total protein concentration was measured using Pierce BCA protein assay kit in accordance to the manufacturer's instructions.

QDB analysis The linear range of a specific antibody (EP3 or 4B5 clone for Her2; MD31 for Ki67, SP1 for Estrogen receptor (ER) and 1E2 for Progesterone receptor, SA38-08 for cyclinD1 were determined by using a pooled lysate from patients testing positive respectively for these biomarkers. The lysates were prepared first by mixing in equal amount tissue lysates prepared from 3 to 4 breast cancer tissues. The pooled lysates were serially diluted from 0-2 □g to define the linear range of QDB analysis. A protein standard either obtained commercially or expressed and purified in the company was also serially diluted from 0-500 pg, and used to define the linear range of QDB analysis.

The samples were applied onto the QDB plates at 2□1/unit in triplicate, and were processed as described previous. A primary antibody was used for primary antibody incubation at 100 ul/well overnight at 4° C. and a donkey anti-rabbit or donkey anti-mouse secondary antibody was incubated with the plate for 4 hours at room temperature. The plates were briefly rinsed twice with TBST, and washed 5×10mins before they were inserted into a white 96-well plate pre-filled with ECL solution prepared according to the manufacturer's instruction at 100□l/well for 3 mins. The chemiluminescence signal from individual well of the recombinant plate was quantified by using the Tecan Infiniti 200 pro Microplate reader with the option “plate with cover”.

The readings were used to measure the biomarker level in FFPE specimens in reference to the protein standard. The measured biomarker levels of PR, ER, Her2 and Ki67 were entered into the database. Samples were divided into three groups for QDB analysis. The consistency of the results were validated by picking 6 samples (2 strong expression, 2 weak expression and 2 medium expression) from each group, and measured in the same experiments.

The results were used to create a 3D scatterplot using OriginPro 9.1 software.

Example 1 teaches how create a 3D scatterplot using protein levels of ER, PR, and Her2 and use this scatterplot to determine the treatment plan for a patient.

The protein levels of PR, ER and Her2 were measured with QDB method as absolute and continuous variables. The results were entered in a QDB database.

Results from more samples were entered into the same QDB database to ensure the growth of the database.

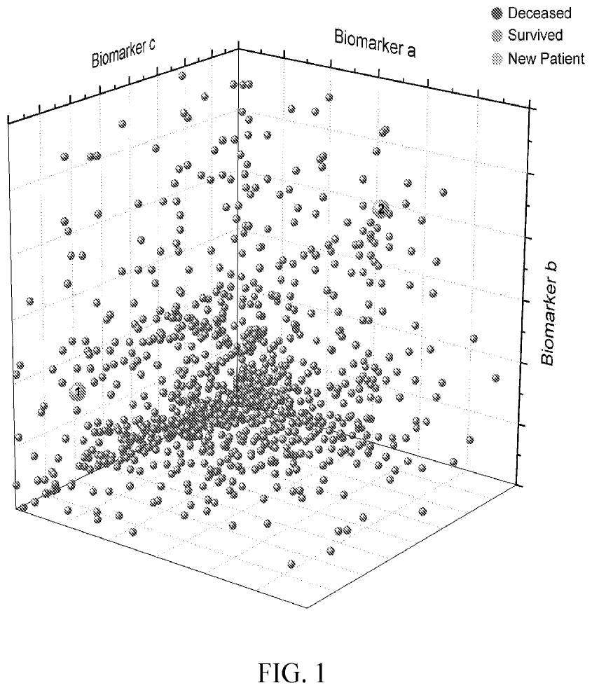

ER, PR and Her2 levels from the QDB database were used to create 3D scatterplot, and this scatterplot is constantly adjusted to ensure the accuracy and comprehensiveness of this scatterplot in FIG. 1.

The scatterplot with each spot representing a sample is defined as reference spatial database.

The clinical information, including DFS and OS, is associated with each spot in the reference spatial database using mathematical analysis.

The ER, PR and Her2 levels from a FFPE specimen from a patient was measured with QDB method.

The spot for this patient is located in the space of reference spatial database.

A Reference spatial profile is identified with the spot of the patient, and the clinical information from this reference spatial profile is analyzed to serve for diagnosis, prediction and prognosis of the patient.

Alternatively, the reference spatial profiles were grouped by their spatial localization into difference sub-groups.

The clinical information, including the DFS and OS, was associated with each sub-group of reference spatial profiles. In this case, the hormone group, Her2 group and Corner group.

ER, PR and Her2 levels of a FFPE specimen from a patient were measured with QDB method, and used to localize the spot with a sub-group by the spatial localization of the spot.

The clinical diagnosis, prediction and prognosis is provided for the patient based on the sub-group where the spot is located.

### EXAMPLE 2

The details of MATERIALS AND METHODS are described in Example 1.

This example teaches how to use 3D model based on the clinical studies to provide diagnosis, prediction and prognosis of a patient.

A 3D model relating the spatial localization to clinical information, including DFS and OS, is developed by analysis of a population of research spatial profiles with the matching clinical informations.

The protein levels of three biomarkers of a patient are determined as absolute and continuous variables.

A spatial localization of a patient based on the expression levels of three biomarkers is assigned in the 3D model supported by an apparatus or a software.

A diagnosis, prediction or prognosis is provided by the 3D model based on the spatial location of the patient determined by three measured biomarkers.

### EXAMPLE 3

The details of MATERIALS AND METHODS are described in Example 1.

This example teaches how to use two 3D scatterplots sequentially to sub-sub-group patients for clinical diagnosis, prediction and prognosis.

Six biomarkers, including ER, PR, Her2, ki67, PCNA and p53 were measured using FFPE specimens from two patient with QDB method.

The spots determined by the expression levels of ER, PR and Her2 from the two patient were assigned to the same sub-group based on the spatial location in the 3D scatterplot of reference spatial database using ER, PR and Her2 as X, Y and Z axes.

Two different spots were determined by the expression levels of Ki67, PCNA and p53 from these two patients in the 3D scatterplot using Ki67, PCNA and p53 as X, Y and Z axes.

Spot 201 was located on the side wall of Ki67 and PCNA, with no expression of p53, while spot 202 was floating in the space with strong expressions of Ki67, PCNA and p53.

Thus, although patient 201 and 202 belongs to the same sub-group (luminal A group) in 3D scatterplot by ER, PR and Her2, they were in the different sub-subgroup in the 3D scatterplot by Ki67, PCNA and p53.

### EXAMPLE 4

A study was conducted using a FFPE breast cancer profile database developed from 427 FFPE specimens collected locally. The clinical outcome was limited to overall survival (OS) of the patients in this study. While the optimized set of protein biomarkers used for assessing similarity remains to be explored, several commonly used breast cancer biomarkers (ER, PR, Her2, Ki67 and cyclinD1) were measured absolutely and quantitatively using Quantitative Dot Blot (QDB) method in all these FFPE specimens. The measured levels of these protein biomarkers, in combination with documented clinicopathological factors (age, tumor size, tumor grade, node status), treatment received and the resulted clinical outcomes, create a ICP for each specimen in this primitive cancer profile database.

Five FFPE profiles (#1388, #1843, #1445, #1807, and #1519) with at least one biomarker level drastically different from each other, were randomly picked from the cancer profile database to be used as hypothetical new patients (Table 1). Incidentally, they also represented the four clinical subtypes based on IHC-based surrogate assay5, with #1388 and #1843 as Triple Negative (TNBC), #1445 as Her2 positive, #1807 as Luminal A-like and #1519 as Luminal B-like.

ER, PR, Her2 and Ki67, the four biomarkers were first used to define the clinical subtype of a patient in daily clinical practice5, to assess the similarity of hypothetical patients with every cancer profile in the database respectively. The Euclidean distances between a hypothetical patient with every cancer profile in the database were calculated and ranked from the lowest to highest. The expression levels of a biomarker the same were considered if they were below the limit of Quantitation (LOQ). In addition, due to the small size of the database, cancer profiles with one or more biomarker levels <50% or 2 fold above that of the hypothetical patient were rejected. For example, for #1519 with ER level at 2 nmole/g, any cancer profiles with ER level <1 or >4 nmol/g were rejected.

18 qualified profiles for #1388, 35 profiles for #1843, 10 profiles for #1445, 14 profiles for #1807, and only 3 cancer profiles for #1519 in this small database (Table 1) were found. Clearly, the size of the database significantly limited our analytical capacity, which further emphasized the need to expand the database to tens of thousands, even to millions, of cancer profiles before the full potential of this method can be achieved.

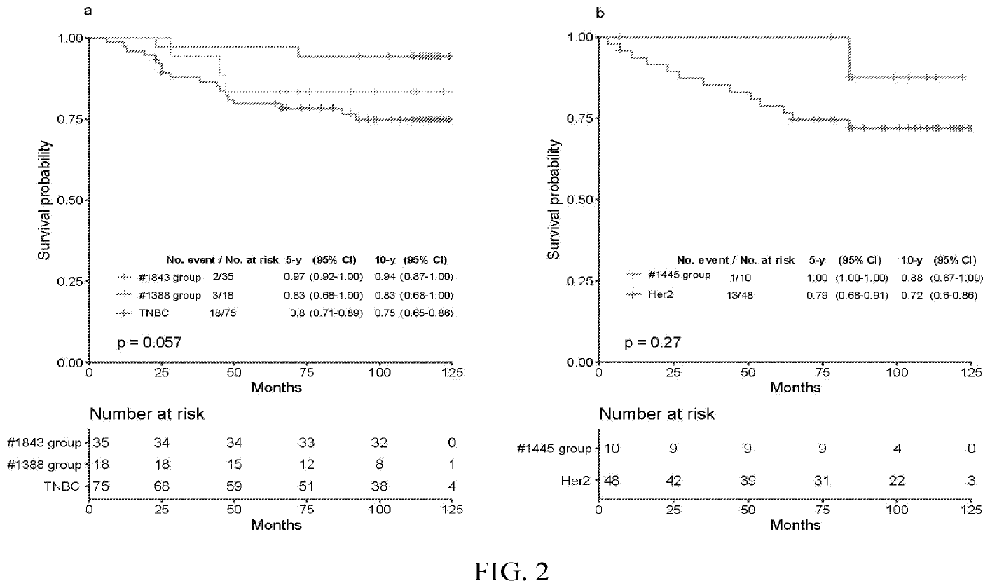

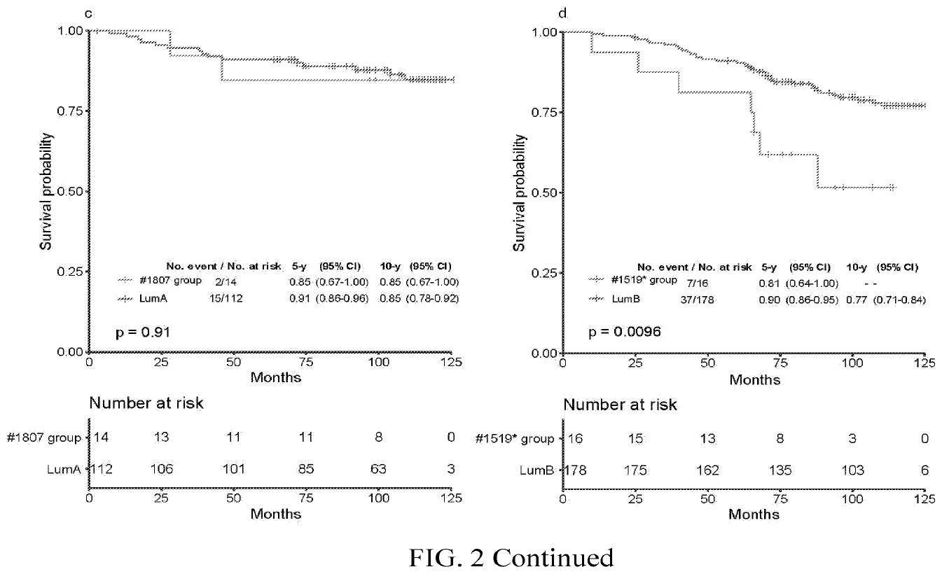

The OS of each group of profiles of similarity (similarity group) was compared with that of the corresponding clinical subtype of the hypothetical patient (FIG. 2). As shown in FIG. 2a, both #1388 and #1843 groups showed slightly better 10 year survival probability (10y SP) than that of the TNBC subtype. However, these differences had not reached statistical significance yet (p=0.057). For #1445, a Her2 positive subtype, its similarity group showed slightly improved OS than that of overall Her2 positive subtype, with 10y SP improved from 72% to 88% (p=0.2′7). For the 14 profiles similar to #1807, a Luminal A-like subtype, their 10y SP were highly similar to that of overall Luminal A-like subtype (p=0.91).

3 profiles similar to #1519, a Luminal B-like profile due to the small size of the database were found. To extract potential indicative information from this dataset for reference, the limitations were relaxed slightly by including profiles with biomarker levels less than 2xLOQ. For example, the LOQ of Her2 was 0.15 nmole/g. Any profiles with Her2 levels <0.3 nmole/g were at same level. With this relaxed requirements, 16 profiles were identified with significantly worsen 10y SP than that of Luminal B-like subtype (p=0.0096).

Conceivably, the more biomarkers are included in the assessment, the higher similarity may be achieved between a profile and the new patient. Therefore, cyclinD1, a biomarker found to be independent from Ki67 in predicting OS of Luminal-like patients6 was included to identify the respective similarity group of these 5 hypothetical patients. Again, the Euclidean distances were calculated for each profile in the database to the 5 hypothetical patients respectively, and ranked by their distance (Table 2). Profiles with any biomarker level <50% or more than 2 fold over that of the hypothetical patient were also rejected.

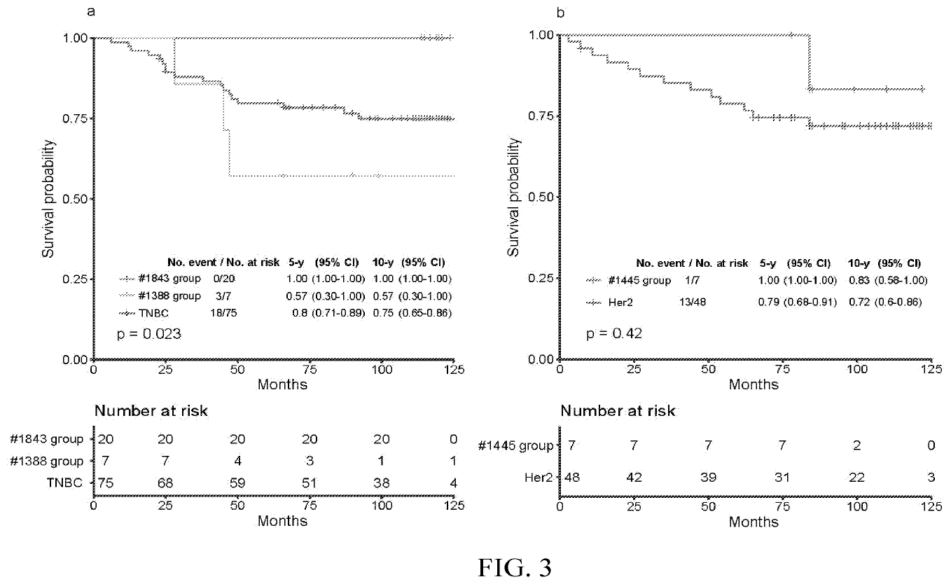

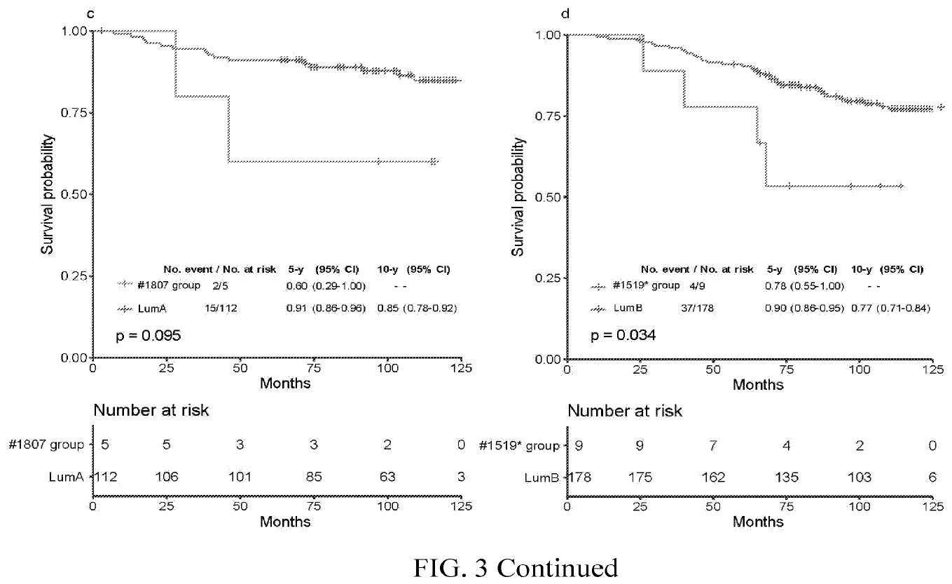

As expected, fewer profiles of similarity to these hypothetical patients in all the cases were found, with #1388 group decreased from 18 to 7 profiles, #1843 group from 35 to 20 profiles, #1445 group from 10 to 7 profiles, #1807 group from 14 to 5 profiles, and #1519 group from 16 to 9 profiles with relaxed rules. The OS of each similarity group was also compared using Log Rank test with that of corresponding clinical subtype of the hypothetical patient (FIG. 3). Unexpectedly, the inclusion of cyclinD1 was able to separate both the #1843 group and #1388 group from the TNBC group to reach statistical level (p=0.023), with significantly better 10y SP for #1843 group at 100%, and significantly worse 10y SP at 57% for #1388 than that of the TNBC subtype at 75% (FIG. 3a). On the other hand, the addition of cyclin D1 offered little help to the prognosis of #1445 (FIG. 3b). The inclusion of cyclinD1 showed a worsen prognosis for #1807 group than that of Luminal A-like subtype, yet this difference had not reach statistical difference (p=0.095). For #1519 group, their OS remained worse than that of Luminal B-like subtype, with p=0.034 from Log Rank test (FIG. 3d).

the most useful application of this method is to provide tailored treatment recommendation for the new patient is considered. Thus, the profiles within each similarity group were further stratified by the treatments they received, and OS analysis was performed to identify the treatment with the best outcome. Due to small size of the database, the treatments were categorized into Chemotherapy alone (Chemo), Endocrine therapy alone (ET), and chemoendocrine therapy (CET). Similarity group identified based on four biomarkers of ER, PR, Her2 and Ki67 were used in this study to include more profiles for analysis.

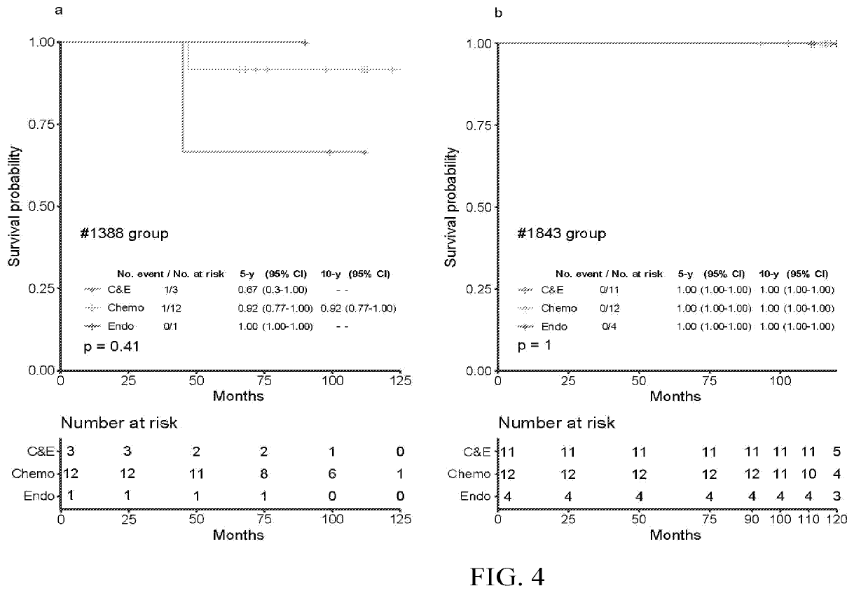

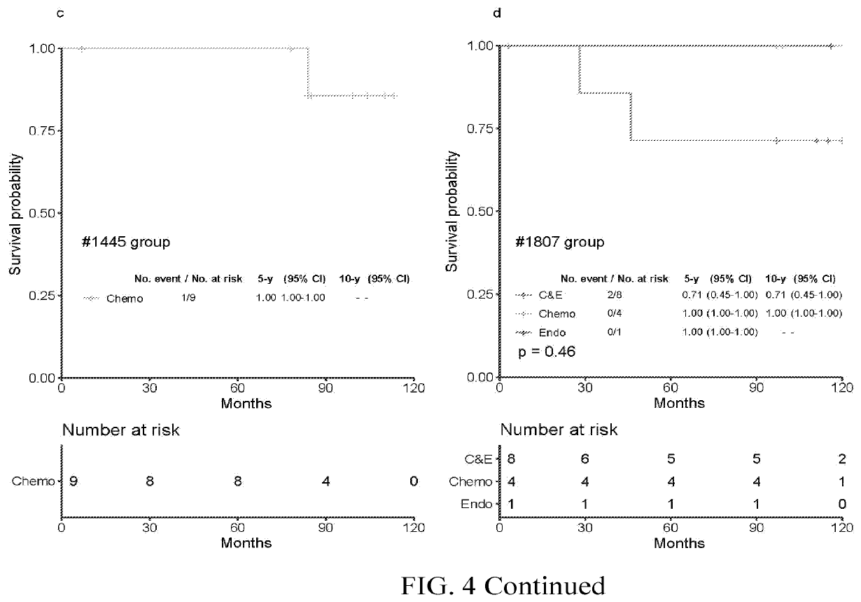

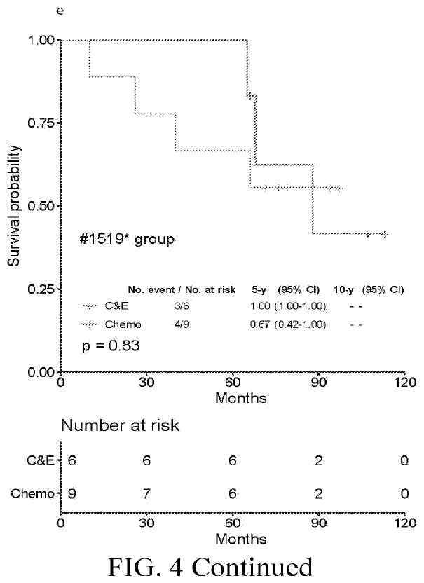

For the #1388 group (FIG. 4a), patients receiving Chemo alone (n=12) had 5y SP at 92%, in comparison to the 67% for those receiving CET (n=3) were observed. For #1843 group, regardless of the treatments received, all patients survived. For #1445 group, no information could be extracted, as all the patients in this group received Chemo alone. For #1807 group, patients receiving Chemo alone (n=4) had 10y SP at 100% vs 71% for patients receiving CET (n=8). For #1519 group, while the 5y SP of the patients receiving CET (n=6) showed advantage over those receiving Chemo alone (n=9), this advantage disappeared later.

Thus, it is demonstrated here the feasibility of proximity-based diagnostic method to provide personalized OS prediction for the five hypothetical patients. Significantly different 10y SP of #1388, #1843, and #1519 was demonstrated from that of the corresponding clinical subtype. The effectiveness of various treatments to the similarity groups of the five hypothetical patients were also evaluated through OS analysis. Yet, due to limited size of the database, their differences were not statistically significant to offer guidance to these hypothetical patients.

It is noted that although #1843 belonged to TNBC, the clinical subtype with worst prognosis7, all the patients in its similarity group remained alive by the end of the study, regardless of the treatments received (FIG. 3a & FIG. 4b). It was suspected #1843 was the Normal-like subtype described in the original molecular subtyping study8.

Evidently, with both the concept and technique ready, the rate-limiting step in the application of this novel diagnostic method in daily clinical practice is to develop a significantly larger cancer profile database than the one used in this study. For example, with over 10,000 breast cancer profiles, it would be identified that around 500 profiles (10,000/400×20) highly similar to #1843, 125 profiles for #1807, and 175 profiles for both #1388 and #1445 based on all five biomarkers to provide trustworthy guidance to the five hypothetical patients.

Yet, even at this scale, it would still be difficult to identify sufficient number of profiles in similarity to #1519, as only 75 profiles maybe identified based on four biomarkers, and much less if all five biomarkers are used in assessing the similarity. Conceivably, patients like #1519 are more likely to be over- or under-treated under current clinical practice, as they are unlikely to be adequately addressed in a clinical trials with hundreds to thousands of cases.

Likewise, the treatments were categorized in this study into Chemo, ET and C&E due to the limited size of database. However, for Chemotherapy alone, there are at least four types of drugs of alkylating agents, anti-tumor antibiotics, antimetabolites, and mitotic inhibitors. Within each type, there are also several different drugs to choose from. Clearly, the exact drug or drugs with best outcome predictions to the new patient can only be identified with an expanded database.

All these considerations emphasize the need to expand the database extensively. Fortunately, the proposed cancer profile database is a growing database due to its absolute nature3. With the acceptance of this method worldwide, it is expected to grow this database exponentially in the near future.

It is not necessary that the five biomarkers used in this study are the best candidates to assess similarity for breast cancer patients. Rather, it is the basis to find the optimum number and combinations of biomarkers from all the existing biomarkers and biomarker candidates for assessing similarity among breast cancer patients. The minimum number of the profiles of similarity required for trustworthy guidance to the patients will also need to be determined through the collaborate efforts from oncologists and statisticians worldwide.

Admittedly, the Euclidean distance was of limited use at this study, as there were limited number of profiles with all the biomarkers within >50% and <200% of those of the hypothetical patient. However, it is envisioned to be extremely useful with a significantly expanded database, where a cut is made to identify those profiles of highest similarity to the new patient.

It is also worthy of mentioning that although in the current study, the similarity is based entirely on the expression levels of a set of protein biomarkers, other clinicopathological factors, including age, tumor size, node status, even predictor scores like RS score from Oncotype, or ROR score from PAM50, can be incorporated to improve the level of similarity. Likewise, while this study is limited in OS analysis, other clinical outcomes, including recurrence, may be used in the evaluation process whenever applicable.

### Materials and Methods

Human subjects and human cell lines: the total 490 Formalin Fixed Paraffin Embedded (FFPE) breast cancer tissues in 2×15 μm slices, with 427 with overall survival (OS) data, were provided from Yantai Affiliated Hospital of Binzhou Medical University and Affiliated Yantai Yuhuangding Hospital of Qingdao University (Yantai, P. R. China) respectively. All the study, including samples collection and study protocol, were in accordance with the Declaration of Helsinki, and were approved by the Medical Ethics Committee of Yantai Affiliated Hospital of Binzhou Medical University (Approval #: 20191127001) to J. Hao6,10, and were approved by ethics committee of Affiliated Yantai Yuhuangding Hospital of Qingdao University ([2017]76 to Guohua Yu)4. In both studies, informed consent were waived for anonymized archival tissues with retrospective clinical data.

All the clinical information was collected from medical records, except biomarker levels, which was measured by Quantitative Dot Blot (QDB) method4,10. The QDB process was also described in detail elsewhere4,10.

General reagents: All the general reagents were described elsewhere4, with anti-ER (SP1) rabbit monoclonal primary antibody was purchased from Abcam Inc, anti-PR (1E2) rabbit monoclonal primary antibody were purchased from Roche Diagnostics GmbH, and anti-Her2(EP3) rabbit monoclonal primary antibody, anti-Ki67 (MD31) mouse monoclonal primary antibody and anti-cyclinD1 rabbit monoclonal antibody (EP12) were purchased from ZSGB-BIO (www.zsbio.com, Beijing, China). HRP labeled Donkey Anti-Rabbit IgG secondary antibody was purchased from Jackson Immunoresearch lab (Pike West Grove, PA, USA). QDB plate was manufactured by Quanticision Diagnostics Inc at RTP, NC, USA.

Calculation of Euclidean distance: the distance between sample a (ER, PR, Her2, Ki67) and b (ER′, PR′, Her2′, Ki67′), and between sample c (ER, PR, Her2, Ki67 & cyclinD1) and d (ER′, PR′, Her2′, Ki67′, cyclinD1′) were calculated using formula:

d_ab=√((ER-ER′)\textcircled{ }2+(PR-PR′)\textcircled{ }2+(Her2-Her2′)\textcircled{ }2+(Ki67-Ki67′)\textcircled{ }2), and

d_cd=√(ER-ER′)\textcircled{ }2+(PR-PR′)\textcircled{ }2+(Her2-Her2′)\textcircled{ }2+(Ki67-Ki67′)\textcircled{ }2+(cyclinD1-cyclinD1′)\textcircled{ }2) .

QDB results of each biomarkers beforehand in all samples (using “normal z-score” transformations) were normalized. The Limit of Quantitation(LOQ) was 0.15 nmol/g for Her2, 0.1 nmol/g for ER, 0.25 nmol/g for PR, and 1.3 nmol/g for Ki67.

Survival analysis: Overall survival of different subgrouping was visualized by Kaplan-Meier method 11, and comparisons were performed by log-rank test. P values of <0.05 were considered statistically significant. All statistical analyses were carried out using R 4.0.1 (http://www.r-project.org).

It is believed that the following claims particularly point out certain combinations and subcombinations that are directed to one of the disclosed inventions and are novel and nonobvious. Inventions embodied in other combinations and subcombinations of features, functions, elements and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

TABLE 1
Groups of cancer profiles of similarity (Similarity group) based on the absolute quantitated protein levels of ER, PR, Her2 and Ki67.
Table 1-1 Similarity group of #1388

| Sample | PR | Her2 | ER | ki67 | | Node | Tumor | Histology | | Clinical | Time | | Euclidean |
|---|---|---|---|---|---|---|---|---|---|---|---|---|---|
| # | (nmol/g) | (nmol/g) | (nmol/g) | (nmol/g) | Age | Status | Size | Grade | Treatment | Subtype | (Months) | Event | Distance |
| 1388 | 0.24 | 0.00 | 0.04 | 12.18 | 41 | N1 | T2 | 3 | C&E | TNBC | 99 | 0 | 0.0000 |
| 1516 | 0.12 | 0.00 | 0.07 | 12.23 | 53 | N0 | T2 | Unknown | C | TNBC | 68 | 0 | 0.0582 |
| 1340 | 0.22 | 0.00 | 0.05 | 10.24 | 60 | N0 | T2 | 3 | C&E | TNBC | 112 | 0 | 0.6049 |
| 1356 | 0.09 | 0.02 | 0.06 | 10.22 | 86 | N2 | T3 | 3 | Unknown | TNBC | 28 | 1 | 0.6117 |
| 1887 | 0.18 | 0.00 | 0.04 | 9.49 | 46 | N0 | T1 | 3 | C | TNBC | 112 | 0 | 0.8373 |
| 1477 | 0.09 | 0.02 | 0.04 | 9.30 | 60 | N0 | T1 | 2 | C | TNBC | 76 | 0 | 0.8969 |
| 1299 | 0.11 | 0.00 | 0.04 | 8.51 | 84 | N1 | T3 | 3 | C | TNBC | 47 | 1 | 1.1426 |
| 1281 | 0.12 | 0.00 | 0.04 | 8.46 | 47 | N0 | T2 | 3 | C | TNBC | 130 | 0 | 1.1572 |
| [symbol] | 0.19 | 0.00 | 0.03 | 8.36 | 41 | Unknown | T2 | 3 | C | TNBC | 111 | 0 | 1.1888 |
| [symbol] | 0.08 | 0.02 | 0.05 | 8.03 | 57 | N0 | T2 | 3 | C | LumB | 72 | 0 | 1.2921 |
| [symbol] | 0.17 | 0.06 | 0.05 | 16.37 | 44 | N0 | T2 | 3 | Unknown | TNBC | 111 | 0 | 1.3027 |
| 1524 | 0.08 | 0.00 | 0.04 | 7.65 | 65 | N0 | T2 | 3 | C | TNBC | 66 | 0 | 1.4105 |
| 1426 | 0.06 | 0.00 | 0.06 | 7.47 | 53 | N0 | T1 | 3 | E | TNBC | 90 | 0 | 1.4658 |
| 1685 | 0.23 | 0.00 | 0.05 | 7.28 | 58 | N0 | T2 | 3 | C | TNBC | 122 | 0 | 1.5243 |
| 1881 | 0.18 | 0.00 | 0.04 | 7.08 | 53 | N0 | T1 | 3 | c | TNBC | 113 | 0 | 1.5887 |
| 1335 | 0.16 | 0.05 | 0.04 | 6.75 | 52 | N0 | T2 | 3 | c | TNBC | 113 | 0 | 1.6901 |
| 1395 | 0.19 | 0.00 | 0.03 | 6.05 | 70 | N1 | T1 | 3 | c | TNBC | 98 | 0 | 1.9084 |
| 1508 | 0.13 | 0.00 | 0.07 | 22.21 | 47 | N0 | T2 | 3 | C&E | TNBC | 45 | 1 | 3.1187 |

[symbol]

indicates data missing or illegible when filed

TABLE 1-2
Similarity group of #1843

| Sample | PR | Her2 | ER | ki67 | | Node | Tumor | Histology | Treatment | Clinical | Time | | Euclidean |
|---|---|---|---|---|---|---|---|---|---|---|---|---|---|
| # | (nmol/g) | (nmol/g) | (nmol/g) | (nmol/g) | Age | Status | Size | Grade | | Subtype | (Months) | Event | Distance |
| 1843 | 0.16 | 0.00 | 0.03 | 0.00 | 41 | Unknown | T1 | 3 | Unknown | TNBC | 114 | 0 | 0.0000 |
| 1743 | 0.17 | 0.00 | 0.03 | 0.00 | 41 | N0 | T2 | Unknown | C&E | LumA | 120 | 0 | 0.0047 |
| 1714 | 0.09 | 0.00 | 0.03 | 0.00 | 36 | N0 | T1 | Unknown | Unknown | Unknown | 121 | 0 | 0.0103 |
| 1800 | 0.14 | 0.00 | 0.02 | 0.00 | 50 | N1 | T3 | 3 | C | TNBC | 117 | 0 | 0.0110 |
| 1825 | 0.18 | 0.00 | 0.04 | 0.00 | 47 | N1 | T2 | Unknown | C | Unknown | 115 | 0 | 0.0117 |
| 1745 | 0.18 | 0.00 | 0.04 | 0.00 | 55 | N0 | T1 | Unknown | Unknown | TNBC | 119 | 0 | 0.0120 |
| 1845 | 0.15 | 0.00 | 0.04 | 0.00 | 73 | Unknown | T1 | 2 | C | TNBC | 114 | 0 | 0.0125 |
| 1795 | 0.06 | 0.00 | 0.03 | 0.00 | 36 | N0 | T1 | 2 | C | TNBC | 117 | 0 | 0.0159 |
| 1708 | 0.05 | 0.00 | 0.03 | 0.00 | 32 | N1 | T1 | 2 | C&E | LumB | 121 | 0 | 0.0178 |
| 1715 | 0.04 | 0.00 | 0.03 | 0.00 | 70 | N0 | T2 | 2 | E | LumB | 120 | 0 | 0.0188 |
| 1706 | 0.05 | 0.00 | 0.04 | 0.00 | 24 | N2 | T1 | Unknown | C&E | LumB | 121 | 0 | 0.0192 |
| 1724 | 0.04 | 0.01 | 0.03 | 0.00 | 31 | N0 | T2 | 1 | C&E | LumA | 120 | 0 | 0.0193 |
| 1709 | 0.17 | 0.00 | 0.04 | 0.00 | 34 | N0 | T2 | 1 | C&E | LumA | 121 | 0 | 0.0216 |
| [symbol] | 0.18 | 0.00 | 0.04 | 0.00 | 57 | N0 | T1 | Unknown | Unknown | LumB | 119 | 0 | 0.0226 |
| | 0.21 | 0.00 | 0.05 | 0.00 | 40 | Unknown | T1 | 1 | C&E | LumA | 116 | 0 | 0.0301 |
| 1734 | 0.23 | 0.00 | 0.05 | 0.00 | 52 | N0 | T1 | 2 | C&Unknown | LumB | 120 | 0 | 0.0354 |
| 1732 | 0.15 | 0.09 | 0.03 | 0.00 | 55 | N0 | T1 | 2 | C | TNBC | 120 | 0 | 0.0367 |
| 1742 | 0.14 | 0.00 | 0.06 | 0.00 | 56 | N0 | T1 | 2 | C | Unknown | 120 | 0 | 0.0503 |
| 1927 | 0.18 | 0.00 | 0.06 | 0.00 | 41 | N0 | T1 | 2 | E | LumB | 111 | 0 | 0.0508 |
| 1704 | 0.20 | 0.00 | 0.06 | 0.00 | 57 | N0 | T1 | 2 | C | Her2 | 121 | 0 | 0.0531 |
| 1305 | 0.07 | 0.00 | 0.06 | 0.00 | 51 | N1 | T1 | 2 | C | LumB | 124 | 0 | 0.0537 |
| 1844 | 0.16 | 0.15 | 0.03 | 0.00 | 46 | Unknown | T2 | 3 | C | Her2 | 114 | 0 | 0.0579 |
| 1380 | 0.14 | 0.08 | 0.06 | 0.00 | 52 | N1 | T2 | 3 | C | LumB | 103 | 0 | 0.0626 |
| 1821 | 0.14 | 0.00 | 0.07 | 0.00 | 45 | N1 | T1 | 2 | C&E | LumB | 115 | 0 | 0.0630 |
| 1776 | 0.19 | 0.00 | 0.07 | 0.00 | 48 | N0 | T1 | 1 | C&E | LumA | 118 | 0 | 0.0680 |
| 1841 | 0.16 | 0.00 | 0.07 | 0.00 | 55 | N0 | T1 | 2 | C&Unknown | LumA | 23 | 1 | 0.0730 |
| 1779 | 0.13 | 0.00 | 0.07 | 0.00 | 53 | Unknown | T2 | 2 | Unknown | LumB | 72 | 1 | 0.0744 |
| 1754 | 0.16 | 0.00 | 0.08 | 0.00 | 47 | N0 | T1 | 1 | C&Unknown | LumA | 119 | 0 | 0.0756 |
| 1707 | 0.06 | 0.00 | 0.08 | 0.00 | 43 | Unknown | T2 | 1 | E | LumA | 121 | 0 | 0.0791 |
| 1705 | 0.17 | 0.00 | 0.08 | 0.00 | 60 | N0 | T1 | 1 | E | LumA | 121 | 0 | 0.0909 |
| 1823 | 0.16 | 0.00 | 0.09 | 0.00 | 43 | Unknown | T1 | 1 | C&E | LumA | 115 | 0 | 0.0938 |
| 1341 | 0.19 | 0.04 | 0.09 | 0.00 | 31 | N2 | T2 | 2 | C&E | LumA | 112 | 0 | 0.0955 |
| 1323 | 0.11 | 0.00 | 0.09 | 0.00 | 40 | N1 | T1 | 2 | C | LumA | 117 | 0 | 0.1046 |
| 1413 | 0.10 | 0.02 | 0.09 | 1.05 | 56 | N1 | T2 | 2 | C | TNBC | 93 | 0 | 0.3439 |
| 1329 | 0.07 | 0.09 | 0.02 | 1.21 | 44 | N0 | T2 | 3 | C&E | TNBC | 116 | 0 | 0.3801 |

[symbol]

indicates data missing or illegible when filed

TABLE 1-3
Similarity group of #1445

| Sample | PR | Her2 | ER | ki67 | | Node | Tumor | Histology | | Clinical | Time | | Euclidean |
|---|---|---|---|---|---|---|---|---|---|---|---|---|---|
| # | (nmol/g) | (nmol/g) | (nmol/g) | (nmol/g) | Age | Status | Size | Grade | Treatment | Subtype | (Months) | Event | Distance |
| 1445 | 0.17 | 1.35 | 0.07 | 4.53 | 58 | N0 | T1 | 2 | C | Her2 | 84 | 0 | 0.0000 |
| [symbol] | 0.10 | 1.36 | 0.04 | 3.52 | 52 | N1 | T1 | 2 | C | Her2 | 78 | 0 | 0.3172 |
| [symbol] | 0.14 | 1.03 | 0.03 | 5.67 | 56 | N0 | T1 | 3 | C | Her2 | 113 | 0 | 0.3835 |
| [symbol] | 0.11 | 1.87 | 0.07 | 3.30 | 61 | N0 | T2 | 3 | C | Her2 | 104 | 0 | 0.4312 |
| 1697 | 0.19 | 1.49 | 0.05 | 3.13 | 52 | N0 | T2 | 3 | Unknown | Her2 | 122 | 0 | 0.4387 |
| 1389 | 0.21 | 2.27 | 0.09 | 3.25 | 54 | N1 | T1 | 2 | C | LumB | 99 | 0 | 0.5359 |
| 1425 | 0.08 | 2.04 | 0.05 | 3.01 | 58 | N2 | T2 | 2 | C | Her2 | 84 | 1 | 0.5451 |
| 1695 | 0.17 | 0.76 | 0.04 | 2.75 | 47 | N3 | T2 | 2 | C | Her2 | 7 | 0 | 0.6016 |
| 1346 | 0.10 | 0.89 | 0.05 | 2.33 | 45 | N0 | T1 | 3 | C | Her2 | 110 | 0 | 0.7086 |
| 1442 | 0.10 | 2.42 | 0.08 | 2.54 | 61 | N2 | T1 | 2 | C | Her2 | 85 | 0 | 0.7441 |

[symbol]

indicates data missing or illegible when filed

TABLE 1-4
Similarity group of #1807

| Sample | PR | Her2 | ER | ki67 | | Node | Tumor | Histology | | Clinical | Time | | Euclidean |
|---|---|---|---|---|---|---|---|---|---|---|---|---|---|
| # | (nmol/g) | (nmol/g) | (nmol/g) | (nmol/g) | Age | Status | Size | Grade | Treatment | Subtype | (Months) | Event | Distance |
| 1807 | 1.63 | 0.00 | 0.12 | 0.00 | 48 | N0 | T1 | 1 | E | LumA | 116 | 0 | 0.0000 |
| 1809 | 1.29 | 0.00 | 0.10 | 0.00 | 48 | N1 | T1 | Unknown | C | LumA | 116 | 0 | 0.0676 |
| 1307 | 1.94 | 0.02 | 0.15 | 0.00 | 47 | N0 | T2 | 2 | C | LumA | 123 | 0 | 0.0724 |
| 1908 | 1.40 | 0.00 | 0.17 | 0.00 | 39 | N1 | T1 | Unknown | C&E | LumB | 111 | 0 | 0.0827 |
| 1752 | 1.42 | 0.00 | 0.07 | 0.00 | 48 | N1 | T1 | 1 | C&Unknown | LumA | 119 | 0 | 0.0971 |
| 1839 | 1.38 | 0.09 | 0.19 | 0.00 | 46 | Unknown | T1 | 1 | C&E | LumA | 115 | 0 | 0.1249 |
| 1398 | 1.00 | 0.03 | 0.17 | 0.00 | 49 | N0 | T2 | Unknown | C | LumA | 97 | 0 | 0.1292 |
| 1905 | 1.06 | 0.03 | 0.06 | 0.00 | 53 | N0 | T1 | 2 | C&E | LumA | 3 | 0 | 0.1320 |
| 1392 | 1.36 | 0.02 | 0.21 | 0.00 | 50 | N0 | T1 | 2 | C | LumA | 99 | 0 | 0.1471 |
| 1721 | 0.93 | 0.00 | 0.19 | 0.00 | 48 | N1 | T1 | 1 | C&E | LumA | 120 | 0 | 0.1629 |
| 1396 | 1.14 | 0.00 | 0.21 | 0.00 | 48 | N0 | T1 | 3 | C&E | LumA | 97 | 0 | 0.1671 |
| 1331 | 1.63 | 0.11 | 0.23 | 0.00 | 65 | N3 | T3 | 2 | C&E | [symbol] | 28 | 1 | 0.1869 |
| 1308 | 1.90 | 0.01 | 0.23 | 0.00 | 43 | N0 | T2 | 3 | C&E | LumA | 123 | 0 | 0.1890 |
| 1922 | 1.14 | 0.00 | 0.24 | 0.00 | 55 | N2 | T2 | 2 | C&E | LumA | 46 | 1 | 0.2088 |

TABLE 1-5
Similarity group of #1519*

| Sample | PR | Her2 | ER | ki67 | | Node | Tumor | Histology | | Clinical | Time | | Euclidean |
|---|---|---|---|---|---|---|---|---|---|---|---|---|---|
| # | (nmol/g) | (nmol/g) | (nmol/g) | (nmol/g) | Age | Status | Size | Grade | Treatment | Subtype | (Months) | Event | Distance |
| 1519 | 0.12 | 0.14 | 2.00 | 3.08 | 67 | N2 | T2 | 2 | C&E | LumB | 65 | 1 | 0.0000 |
| 1337 | 0.43 | 0.00 | 2.06 | 2.70 | 65 | N1 | T2 | 3 | C | LumB | 66 | 1 | 0.1729 |
| 1525 | 0.25 | 0.00 | 2.12 | 3.09 | 50 | N0 | T2 | 2 | C&E | LumA | 66 | 0 | 0.2046 |
| 1876 | 0.39 | 0.00 | 1.85 | 1.52 | 68 | N1 | T1 | 1 | C&E | LumB | 113 | 0 | 0.5521 |
| 1397 | 0.36 | 0.00 | 1.63 | 3.61 | 59 | N1 | T2 | 3 | C | LumB | 97 | 0 | 0.6472 |
| 1515 | 0.38 | 0.00 | 1.46 | 2.84 | 59 | N1 | T2 | 3 | C | LumB | 40 | 1 | 0.9191 |
| 1859 | 0.42 | 0.10 | 2.56 | 4.57 | 65 | N1 | T1 | 3 | C&E | LumB | 88 | 1 | 1.0527 |
| 1441 | 0.27 | 0.00 | 1.44 | 5.42 | 53 | N1 | T1 | 2 | C&E | LumB | 68 | 1 | 1.1920 |
| 1358 | 0.21 | 0.21 | 1.40 | 5.24 | 49 | N1 | T2 | 3 | C&E | TNBC | 107 | 0 | 1.2135 |
| 1409 | 0.16 | 0.00 | 1.30 | 4.76 | 61 | N1 | T2 | 3 | C | LumB | 94 | 0 | 1.2918 |
| 1478 | 0.32 | 0.04 | 1.25 | 1.59 | 55 | N1 | T2 | 2 | C | LumA | 76 | 0 | 1.3469 |
| 1858 | 0.37 | 0.00 | 1.21 | 2.25 | 49 | N0 | T1 | 2 | C&Unknown | LumB | 114 | 0 | 1.3478 |
| 1479 | 0.30 | 0.03 | 1.05 | 5.17 | 62 | N3 | T2 | 2 | C | LumB | 10 | 1 | 1.7275 |
| 1464 | 0.37 | 0.01 | 2.95 | 5.29 | 63 | N1 | T2 | 3 | C | LumB | 79 | 0 | 1.7354 |
| 1483 | 0.49 | 0.00 | 1.08 | 5.83 | 56 | N1 | T2 | 3 | C | LumB | 26 | 1 | 1.7638 |
| 1501 | 0.13 | 0.05 | 1.05 | 5.79 | 42 | N0 | T1 | 2 | C | LumB | 71 | 0 | 1.8057 |

Abbreviations: All the clinicopathological parameters are in accordance with the definitions by American Joint Committee on Cancer (AJCC) . C, Chemotherapy; E, Endotherapy; LumA & LumB, Luminal A-like & B-like clinical subtype; TNBC, Triple Negative Breast Cancer; Her2, Her2 positive subtype; Event: 0-alive, 1-deceased.
Note:
All the biomarker levels within the Limit of Quantitation (LOQ) were regarded as the same levels. Samples with at least one biomarker levels significantly different from the hypothetical patient (<50% or >200%) were discarded. * , biomarker levels within 2XLOQ were considered the same levels. Samples with at least one biomarker levels significantly different from the hypothetical patient (<50% or >200%) were discarded.

TABLE 2
Gr [symbol] of cancer profiles of similarity (Similarity group) based on the absolute quantitated protein levels of ER, PR, Her2, Ki67 & cyclinD1
Table 2-1 Similarity group of #1388

| Sample | PR | Her2 | ER | ki67 | cyclinD1 | | Node | Tumor | Histology | | Clinical | Time | | Euclidean |
|---|---|---|---|---|---|---|---|---|---|---|---|---|---|---|
| # | (nmol/g) | (nmol/g) | (nmol/g) | (nmol/g) | (nmol/g) | Age | Status | Size | Grade | Treatment | Subtype | (months) | Event | Distance |
| 1388 | 0.24 | 0.00 | 0.04 | 12.18 | 0.04 | 41 | N1 | T2 | 3 | C&E | TNBC | 99 | 0 | 0.0000 |
| 1356 | 0.09 | 0.02 | 0.06 | 10.22 | 0.05 | 86 | N2 | T3 | 3 | Unknown | TNBC | 28 | 1 | 0.6117 |
| 1299 | 0.11 | 0.00 | 0.04 | 8.51 | 0.01 | 84 | N1 | T3 | 3 | C | TNBC | 47 | 1 | 1.1434 |
| 1281 | 0.12 | 0.00 | 0.04 | 8.46 | 0.08 | 47 | NO | T2 | 3 | C | TNBC | 130 | 0 | 1.1582 |
| 1524 | 0.08 | 0.00 | 0.04 | 7.65 | 0.09 | 65 | NO | T2 | 3 | C | TNBC | 66 | 0 | 1.4115 |
| 1426 | 0.06 | 0.00 | 0.06 | 7.47 | 0.02 | 53 | NO | T1 | 3 | E | TNBC | 90 | 0 | 1.4661 |
| 1508 | 0.13 | 0.00 | 0.07 | 22.21 | 0.10 | 47 | NO | T2 | 3 | C&E | TNBC | 45 | 1 | 3.1194 |

[symbol]

indicates data missing or illegible when filed

TABLE 2-2
Similarity group of #1843

| Sample | PR | Her2 | ER | ki67 | cyclinD1 | | Node | Tumor | Histology | | Clinical |
|---|---|---|---|---|---|---|---|---|---|---|---|
| # | (nmol/g) | (nmol/g) | (nmol/g) | (nmol/g) | (umol/g) | Age | Status | Size | Grade | Treatment | Subtype |
| [symbol] | 0.16 | 0.00 | 0.03 | 0.00 | 0.00 | 41 | Unknown | T1 | 3 | Unknown | TNBC |
| [symbol] | 0.17 | 0.00 | 0.03 | 0.00 | 0.01 | 41 | N0 | T2 | Unknown | C&E | LumA |
| [symbol] | 0.15 | 0.00 | 0.04 | 0.00 | 0.01 | 73 | Unknown | T1 | 2 | C | TNBC |
| 1745 | 0.18 | 0.00 | 0.04 | 0.00 | 0.01 | 55 | N0 | T1 | Unknown | Unknown | TNBC |
| 1708 | 0.05 | 0.00 | 0.03 | 0.00 | 0.01 | 32 | N1 | T1 | 2 | C&E | LumB |
| 1706 | 0.05 | 0.00 | 0.04 | 0.00 | 0.01 | 24 | N2 | T1 | Unknown | C&E | LumB |
| 1825 | 0.18 | 0.00 | 0.04 | 0.00 | 0.02 | 47 | N1 | T2 | Unknown | C | Unknown |
| 1724 | 0.04 | 0.01 | 0.03 | 0.00 | 0.02 | 31 | N0 | T2 | 1 | C&E | LumA |
| 1715 | 0.04 | 0.00 | 0.03 | 0.00 | 0.02 | 70 | N0 | T2 | 2 | E | LumB |
| 1714 | 0.09 | 0.00 | 0.03 | 0.00 | 0.03 | 36 | N0 | T1 | Unknown | Unknown | Unknown |
| 1795 | 0.06 | 0.00 | 0.03 | 0.00 | 0.04 | 36 | N0 | T1 | 2 | C | TNBC |
| 1704 | 0.20 | 0.00 | 0.06 | 0.00 | 0.02 | 57 | N0 | T1 | 2 | C | Her2 |
| 1844 | 0.16 | 0.15 | 0.03 | 0.00 | 0.00 | 46 | Unknown | T2 | 3 | C | Her2 |
| 1821 | 0.14 | 0.00 | 0.07 | 0.00 | 0.01 | 45 | N1 | T1 | 2 | C&E | LumB |
| 1800 | 0.14 | 0.00 | 0.02 | 0.00 | 0.06 | 50 | N1 | T3 | 3 | C | TNBC |
| 1732 | 0.15 | 0.09 | 0.03 | 0.00 | 0.05 | 55 | N0 | T1 | 2 | C | TNBC |
| 1742 | 0.14 | 0.00 | 0.06 | 0.00 | 0.05 | 56 | N0 | T1 | 2 | c | Unknown |
| 1750 | 0.18 | 0.00 | 0.04 | 0.00 | 0.06 | 57 | N0 | T1 | Unknown | Unknown | LumB |
| 1305 | 0.07 | 0.00 | 0.06 | 0.00 | 0.06 | 51 | N1 | T1 | 2 | C | LumB |
| 1705 | 0.17 | 0.00 | 0.08 | 0.00 | 0.03 | 60 | N0 | T1 | 1 | E | LumA |
| | | | | | | | | Similarity group of #1843 | | | |
| | | | | | | | | Sample | Time | | Euclidean |
| | | | | | | | | # | (months) | Event | Distance |
| | | | | | | | | [symbol] | 114 | 0 | 0.0000 |
| | | | | | | | | [symbol] | 120 | 0 | 0.0125 |
| | | | | | | | | [symbol] | 114 | 0 | 0.0143 |
| | | | | | | | | 1745 | 119 | 0 | 0.0188 |
| | | | | | | | | 1708 | 121 | 0 | 0.0206 |
| | | | | | | | | 1706 | 121 | 0 | 0.0213 |
| | | | | | | | | 1825 | 115 | 0 | 0.0220 |
| | | | | | | | | 1724 | 120 | 0 | 0.0266 |
| | | | | | | | | 1715 | 120 | 0 | 0.0271 |
| | | | | | | | | 1714 | 121 | 0 | 0.0338 |
| | | | | | | | | 1795 | 117 | 0 | 0.0535 |
| | | | | | | | | 1704 | 121 | 0 | 0.0570 |
| | | | | | | | | 1844 | 114 | 0 | 0.0579 |
| | | | | | | | | 1821 | 115 | 0 | 0.0644 |
| | | | | | | | | 1800 | 117 | 0 | 0.0681 |
| | | | | | | | | 1732 | 120 | 0 | 0.0722 |
| | | | | | | | | 1742 | 120 | 0 | 0.0776 |
| | | | | | | | | 1750 | 119 | 0 | 0.0782 |
| | | | | | | | | 1305 | 124 | 0 | 0.0907 |
| | | | | | | | | 1705 | 121 | 0 | 0.0990 |

[symbol]

indicates data missing or illegible when filed

TABLE 2-3
Similarity group of #1445

| Sample | PR | Her2 | ER | ki67 | cyclinD1 | | Node | Tumor | Histology | | Clinical | Time | | Euclidean |
|---|---|---|---|---|---|---|---|---|---|---|---|---|---|---|
| # | (nmol/g) | (nmol/g) | (nmol/g) | (nmol/g) | (nmol/g) | Age | Status | Size | Grade | Treatment | Subtype | (months) | Event | Distance |
| 1445 | 0.17 | 1.35 | 0.07 | 4.53 | 0.28 | 58 | N0 | T1 | 2 | C | Her2 | 84 | 0 | 0.0000 |
| 1468 | 0.10 | 1.36 | 0.04 | 3.52 | 0.28 | 52 | N1 | T1 | 2 | C | Her2 | 78 | 0 | 0.3172 |
| 1697 | 0.19 | 1.49 | 0.05 | 3.13 | 0.41 | 52 | N0 | T2 | 3 | Unknown | Her2 | 122 | 0 | 0.4647 |
| 1389 | 0.21 | 2.27 | 0.09 | 3.25 | 0.47 | 54 | N1 | T1 | 2 | C | LumB | 99 | 0 | 0.5813 |
| [symbol] | 0.08 | 2.04 | 0.05 | 3.01 | 0.47 | 58 | N2 | T2 | 2 | C | Her2 | 84 | 1 | 0.5883 |
| [symbol] | 0.10 | 0.89 | 0.05 | 2.33 | 0.29 | 45 | N0 | T1 | 3 | C | Her2 | 110 | 0 | 0.7086 |
| [symbol] | 0.10 | 2.42 | 0.08 | 2.54 | 0.22 | 61 | N2 | T1 | 2 | C | Her2 | 85 | 0 | 0.7482 |

[symbol]

indicates data missing or illegible when filed

TABLE 2-4
Similarity group of #1807

| Sample | PR | Her2 | ER | ki67 | cyclinDl | Age | Node | Tumor | Histology | | Clinical | Time | | Euclidean |
|---|---|---|---|---|---|---|---|---|---|---|---|---|---|---|
| # | (nmol/g) | (nmol/g) | (nmol/g) | (nmol/g) | (umol/g) | | Status | Size | Grade | Treatment | Subtype | (months) | Event | Distance |
| 1807 | 1.63 | 0.00 | 0.12 | 0.00 | 0.12 | 48 | NO | T1 | 1 | E | LumA | 116 | 0 | 0.0000 |
| 1839 | 1.38 | 0.09 | 0.19 | 0.00 | 0.19 | 46 | Unknown | T1 | 1 | C&E | LumA | 115 | 0 | 0.1510 |
| 1398 | 1.00 | 0.03 | 0.17 | 0.00 | 0.18 | 49 | NO | T2 | Unknown | C | LumA | 97 | 0 | 0.1520 |
| 1331 | 1.63 | 0.11 | 0.23 | 0.00 | 0.13 | 65 | N3 | T3 | 2 | C&E | [symbol] | 28 | 1 | 0.1878 |
| 1922 | 1.14 | 0.00 | 0.24 | 0.00 | 0.15 | 55 | N2 | T2 | 2 | C&E | LumA | 46 | 1 | 0.2121 |

TABLE 2-5
Similarity group of #1519*

| Sample | PR | Her2 | ER | ki67 | cyclinDl | | Node | Tumor | Histology | | Clinical | Time | | Euclidean |
|---|---|---|---|---|---|---|---|---|---|---|---|---|---|---|
| # | (nmol/g) | (nmol/g) | (nmol/g) | (nmol/g) | (nmol/g) | Age | Status | Size | Grade | Treatment | Subtype | (months) | Event | Distance |
| 1519 | 0.12 | 0.14 | 2.00 | 3.08 | 1.39 | 67 | N2 | T2 | 2 | C&E | LumB | 65 | 1 | 0.0000 |
| 1397 | 0.36 | 0.00 | 1.63 | 3.61 | 1.64 | 59 | N1 | T2 | 3 | C | LumB | 97 | 0 | 0.7168 |
| 1525 | 0.25 | 0.00 | 2.12 | 3.09 | 0.72 | 50 | N0 | T2 | 2 | C&E | LumA | 66 | 0 | 0.8166 |
| 1515 | 0.38 | 0.00 | 1.46 | 2.84 | 1.11 | 59 | N1 | T2 | 3 | C | LumB | 40 | 1 | 0.9757 |
| 1858 | 0.37 | 0.00 | 1.21 | 2.25 | 0.93 | 49 | N0 | T1 | 2 | C&Unknown | LumB | 114 | 0 | 1.4552 |
| 1478 | 0.32 | 0.04 | 1.25 | 1.59 | 0.91 | 55 | N1 | T2 | 2 | C | LumA | 76 | 0 | 1.4604 |
| 1441 | 0.27 | 0.00 | 1.44 | 5.42 | 2.41 | 53 | N1 | T1 | 2 | C&E | LumB | 68 | 1 | 1.7101 |
| 1358 | 0.21 | 0.21 | 1.40 | 5.24 | 2.47 | 49 | N1 | T2 | 3 | C&E | TNBC | 107 | 0 | 1.7691 |
| 1483 | 0.49 | 0.00 | 1.08 | 5.83 | 1.59 | 56 | N1 | T2 | 3 | C | LumB | 26 | 1 | 1.7799 |

Abbreviations: All the clinicopathological parameters are in accordance with American Joint Committee on Cancer (AJCC). C, Chemotherapy; E, Endotherapy; LumA & Lui [symbol] Luminal A-like & B-like clinical subtype; TNBC, Triple Negative Breast Cancer; Her2, Her2 positive subtype; Event: 0-alive, 1-deceased. Not [symbol] the biomarker levels within the Limit of Quantitation (LOQ) were regarded as the same levels. Samples with at least one biomarker levels significantly different fro [symbol] hypothetical patient (<50% or >200%) were discarded. *, biomarker levels within 2XLOQ were considered the same levels. Samples with at least one biomarker levels significantly different from the hypothetical patient (<50% or >200%) were discarded. [symbol]
indicates data missing or illegible when filed

1. A method of generating a database to provide diagnosis, prediction, or prognosis of cancer, comprising:
providing a plurality of subjects each having a known clinical outcome of a cancer;
generating an individual cancer profile (ICP) from each of the plurality of subjects, the individual cancer profile comprising i) a plurality of clinical parameters that each represents a quantitative measurement of a biomarker, wherein the quantitative measurement is from an archived FFPE specimen, continuous, and an absolute amount of the biomarker in the specimen, and ii) a known clinical outcome of a cancer; and
storing the generated individual cancer profiles of the plurality of subjects in the database.

2. The method according to claim 1, wherein the biomarker is a protein marker.

3. The method according to claim 1, wherein the quantitative measurement is conducted by a quantitative dot blot (QDB).

4. The method according to claim 1, wherein the cancer is breast cancer and the biomarker is Estrogen receptor (ER), Progesterone Receptor (PR), Ki67, p53, cyclinD1, or Her2.

5. A database for providing diagnosis, prediction, or prognosis of cancer comprising a plurality of individual cancer profiles, each generated from a subjects having a known clinical outcome of a cancer, wherein:
an individual cancer profiles comprises i) a plurality of clinical parameters quantitatively measured from an archived FFPE specimen of the subject and ii) a known clinical outcome of a cancer;
each of the plurality of clinical parameters represents a quantitative measurement of a biomarker and
the quantitative measurement is continuous and is an absolute amount of the biomarker in the specimen.

6. The method according to claim 1, wherein the quantitative measurement is conducted by a quantitative dot blot (QDB).

7. The method according to claim 1, wherein the cancer is breast cancer and the biomarker is Estrogen receptor (ER), Progesterone Receptor (PR), Ki67, p53, cyclinD1, or Her2.

8. An apparatus for diagnosing cancer in a patient, the apparatus comprising the database of claim 5.

9. A kit for diagnosing cancer in a patient, the kit comprising the database of claim 5.

10. A method of providing diagnosis, prediction, or prognosis of cancer in a patient, comprising
1) collecting a FFPE specimen of the patient;
2) obtaining from a database of claim 5: i) the stored individual cancer profiles and ii) the set of clinical parameters used in the database;
3) comparing the quantitative level of the set of clinical parameters in the database with those measured from the FFPE specimen of the patient;
4) identifying an individual cancer profile from the database that best matches the patient based on the comparison;
5) outputting a clinical outcome of the identified individual cancer profile from the database.

11. The method according to claim 10, wherein the comparison is to determine maximum similarity between the set of clinical parameters of an individual cancer profile and those of the same set measured from the FFPE specimen of the patient.

12. The method according to claim 11, wherein, when the similarity is measured, the absolute level of each biomarker is within a pre-set range of that of the same biomarker from the same set measured from the FFPE specimen of the patient.

13. The method according to claim 11, wherein the similarity is calculated based on the Euclidean distance between the two sets of quantitative clinical parameters.

14. The method according to claim 10, wherein the quantitative level of the set of clinical parameters from the FFPE specimen is conducted by a quantitative dot blot (QDB).

15. The method according to claim 10, wherein the cancer is breast cancer and the set of clinical parameters comprise Estrogen receptor (ER), Progesterone Receptor (PR), Ki67, p53, cyclinD1, Her2, or a combination thereof.
